(19) United States
(12) Patent Application Publication
(10) Pub. No.: US 2003/0023197 A1
(43) Pub. Date: Jan. 30, 2003

(54) Shrink wrap bandage

(76) Inventor: Jim Liguori, Irvine, CA (US)

Correspondence Address: Mr Jim Liguori, 4892 Basswood, Irvine, CA, 92612, US

(21) Appl. No.: 10/004,137

(22) Filed: Oct. 26, 2001

Related U.S. Application Data
(63) Continuation-in-part of application No. 09/911,970, filed on Jul. 24, 2001.

(51) Int. Cl. A61F 13/00

(52) U.S. Cl. 602/52; 602/54

(57) ABSTRACT

A shrink-wrap bandage is disclosed, along with methods of applying same. The bandage comprises a shrink film cut and/or shaped to a desired dimension and having material properties which allow the shrink film to undergo a controlled degree of shrinkage upon application of a heat source or other suitable source. The sheet may include a variety of conventional features, such as an absorbent pad, adhesive to facilitate contact with the skin, or apertures to allow air to access the covered wound.

### RELATED APPLICATIONS

The present application is continuation-in-part of co-pending U.S. patent application Ser. No. 09/911,970, filed Jul. 24, 2001, and to which priority is hereby expressly claimed as permitted by law. The entire original disclosure of the above-referenced co-pending application is hereby expressly incorporated herein by reference.

### FIELD OF THE INVENTION

The present invention relates generally to bandages, and more particularly to bandages adapted to be shrink-fitted to accommodate characteristics of the target application area or wound site, including the size, shape, and mobility thereof.

### BACKGROUND

Adhesive bandages are well-known in the art. Generally, most adhesive bandages comprise a plastic backing having inwardly- and outwardly-facing surfaces. Disposed upon the inwardly-facing surface is typically a layer or patch of absorbent material designed to compress against the open wound or sore. Such bandages further typically include a layer of adhesive formed adjacent to or about the periphery of such absorbent material to thus enable the bandage to remain in a fixed position relative the wound to thus provide a protective covering thereover. Exemplary of such adhesive bandages include those sold under the well-known brand names Band-Aid® produced by Johnson and Johnson of Skillman, N.J. and Curad® produced by Beiersdorf, Inc. of Wilton, Conn.

Despite the advantages afforded by conventional adhesive bandages, such as the promotion of rapid healing, wound protection, and the like, the same suffer from numerous drawbacks. Firstly, adhesive bandages necessarily require substantial amounts of adhesive spread out over a sufficiently large surface area to thus enable the same to stick and remain in place around the target wound site. Indeed, such adhesive portion of most adhesive bandages currently in use comprises the major component of such bandages and constitutes a significant portion of the cost associated with manufacture of such a bandage. Moreover, the adhesive component requires complex application, special handling and packaging insofar as the same must be covered with protective wax paper liners and the like to thus enable the layers of adhesive to retain their adhesive properties while in their packaged form.

Notwithstanding the extensive use of adhesives, most adhesive bandages currently in use can and do frequently fail to sufficiently adhere about the wound site. As a result, such bandages detract from the healing process. Such inability to adhere to a specific site on a person's body is exceptionally likely if such site is at an area having a high degree of mobility, such as an elbow, knee, finger, toe, ankle, or other jointed area, which undergo constant articulation, particularly with respect to small children.

Moreover, such adhesive can and frequently does become exposed, leaving a sticky residue that attracts dirt and bacteria, and is likewise problematic when it comes time to remove the same, particularly with respect to digits (i.e., fingers and toes) that can touch and contact numerous surfaces, and are constantly moving. Furthermore, as virtually everyone who has had an adhesive bandage will appreciate, the adhesive can cause substantial pain when removed from a person's body.

U.S. Pat. No. 6,221,447, issued to Munn et al. on Apr. 24, 2001, and entitled “Rubber Products That Shrink Due To The Application Of Energy And Hypo-Allergenic Rubbery Products”, is hereby expressly incorporated herein by reference. Munn discloses use of rubbery materials that shrink from a first larger size to a second smaller size upon application of heat or its equivalent in the range near normal human body temperature. Such materials are described in Munn as being specifically formed to have a first shape that, after the application of a higher temperature, which is in the range of 94-99° Fahrenheit, conforms to a second, shrunken shape. Such rubbery materials are disclosed for specific uses, most notably condoms, but other uses noted include gloves, catheters, finger cots, oral dental dams, stretchy bandages, and other rubbery items that are desired to be hypo-allergenic.

Although perhaps well-suited for applications such as condoms and catheters, the same are ill-suited for use as bandages. First of all, insofar as such materials have a relatively low transition temperature (i.e., 94-99° Fahrenheit), such materials are exceptionally difficult to apply, let alone able to sufficiently adhere to the surface of the skin or other target area, in warmer climates. In this regard, such rubbery materials will transition prior to even being applied in many instances, oftentimes by merely coming into contact with normal bodily temperature. Such low transition temperature range is further problematic from a shipping standpoint insofar as such materials must be constantly kept and stored at temperatures below the transition temperature, or else such material will prematurely transition to its shrunken operative state prior to their application. Additionally disadvantageous is the fact that such rubbery materials are produced via complex production processes, including precise cross-linking and strictly regulated temperature parameters, that make such materials impractical to mass produce.

Accordingly, there is a substantial need in the art for an improved bandage that more easily and readily adheres to the skin or other target area about a wound than do prior art adhesive bandages. There is further a need in the art for such a bandage that stays adhered to the skin to a much greater degree than do prior art bandages, and further expressly dispenses or substantially minimizes the need for adhesive to be utilized therewith. There is yet further a need for such a bandage that is of relatively low cost, may be readily fabricated from existing, commercially-available materials, is safe and easy to use, and can be mass produced and shipped in an extremely economical manner.

### SUMMARY OF THE INVENTION

The present invention specifically addresses and alleviates the above-identified deficiencies in the art. More specifically, the present invention is directed to a shrink-wrap bandage that is operative to form a neater, tighter and more secure fit about a wound sought to be protected. Such shrink-wrap bandage is further more sanitary, uses little or no adhesive, is water-resistant and far easier and less expensive to manufacture and ship than prior art bandages and the like.

According to the preferred embodiment, the bandage comprises a sheet of polyvinyl chloride (PVC) or polyolifin shrink film that is designed to assume a first expanded configuration, but transition to a second, shrunken configuration when subjected to elevated temperatures that are preferably approximately 120 degrees Fahrenheit or higher. In this regard, the PVC or polyolifin shrink film has material properties such that the film shrinks from about 8-20% when subjected to such elevated temperatures for about two to three seconds. In more highly refined embodiments, the PVC or polyolifin shrink film will undergo approximately 12-14% shrinkage when subjected to at least approximately 115 degrees Fahrenheit and preferably 120 degrees Fahrenheit or higher for approximately two to three seconds. Among such materials suited for such applications include shrink wrap such as 500-degree shrink-wrap having a gauge of 50 or greater. In a more highly preferred embodiment, the shrink-wrap comprises shrink-wrap having a gauge of at least 75.

Still other suitable materials may be used, which exhibit the desirable transition temperatures and other properties as described herein. Such materials may include rubbery materials as described in the Munn patent, if manufactured to have a transition temperature above 100 degrees Fahrenheit, and preferably above approximately 115 degrees. The transition temperature of a particular suitable material, including shrink wrap materials, may be within a range, such as between approximately 105 degrees and 125 degrees, or between approximately 110 degrees and 125 degrees, or between approximately 115 degrees and 125 degrees, etc. It is preferable that the transition temperature is greater than the normal human body temperature, such as for example at least approximately 105 degrees Fahrenheit, to prevent transition of the bandage from the first state to the second shrunken state merely in response to contact with a human body. Similarly, materials with transition temperatures in the higher range may be desirable in certain instances to prevent transition of the bandage from the first state to the second shrunken state merely in response to natural ambient temperature conditions. Accordingly, use of the phrase “shrink film” encompasses not only PVC or polyolifin, but all such materials which exhibit the desired transition temperatures and other properties as described herein, many of which are known in the art.

In further refinements of the present invention, the shrink-wrap bandage may include a plurality of perforations or apertures, as per conventional adhesive bandages, to enable air to access the covered wound sought to be protected, as well as add comfort and prevent perspiration build-up. Such bandage may further include one or more layers of gauze or absorbent material disposed thereon for contacting with and absorbing fluids from such wound, also as per conventional bandages. Such bandage may further include one or more layers of adhesive to increase the ability of the bandage to remain more securely attached about the wound site. As to this latter aspect, however, it is contemplated that substantially minimal amounts of adhesive will be needed, if at all, due to the superior adhesion accomplished via the shrink-wrap properties of the PVC, polyolifin, or other shrink film.

With respect to the methods of the present invention, there is provided the initial step of placing a bandage of the aforementioned variety over or about the wound sought to be protected wherein the bandage is in its first expanded configuration. While positioned about or over such wound, a transition source (such as a heat source) is applied to such bandage in a degree sufficient to cause the bandage to transition from its first, expanded configuration to its second, shrunken configuration. In regards to a heat source, for example, the heat source applies heat at least sufficient to raise the temperature of the bandage to its transition temperature. Such heat source may comprise any of a variety of well-known sources in the art, including, but not limited to, a heat lamp or a conventional hair dryer. Additional energizing sources are contemplated, such as light or radiation, which could facilitate transition of the shrink film.

As a consequence, the shrink film undergoes transition from its expanded configuration to its second shrunken, operative configuration. During such transition, the bandage will operatively adhere and become fixed to the tissue about the wound or target area, providing a secure, neat, and tight fit thereabout. Such bandage may thereafter be worn as per conventional bandages and simply removed from the skin following use thereof. Due to its elimination or minimal use of adhesive, as well as the use of PVC or polyolifin, such bandage is water-resistant and exceptionally easy and pain-free to remove. Other suitable materials may also be water resistant or water repellant.

It is therefore an object of the present invention to provide a shrink-wrap bandage that is more substantially effective at adhering to the skin and forming a protective covering about a wound than are prior art adhesive bandages and the like.

Another object of the present invention is to provide a shrink-wrap bandage that uses little or no adhesive, is easier to apply, and easier and less painful to remove than are prior art adhesive bandages and the like.

Another object of the present invention is to provide a shrink-wrap bandage that is easy and simple to manufacture, requires no special packaging, and is substantially less expensive than prior art adhesive bandages and the like.

Still further objects of the present invention are to provide a shrink-wrap bandage that can be selectively sized and dimensioned for use in a wide variety of wound covering applications, need not be manufactured or used under rigorous temperature control, may be manufactured without the need for complex production processes, results in a bandage that is water-resistant, may be readily fabricated from commercially-available materials and may be readily used in place of conventional prior art bandages and the like.

Theses objects may be accomplished either alone or in combination with one another, along with other objects, as described more fully herein.

### BRIEF DESCRIPTION OF THE DRAWINGS

These as well as other features of the present invention will become more apparent upon reference to the drawings wherein:

FIG. 1 is a frontal view of a shrink-wrap bandage constructed in accordance with a preferred embodiment of the present invention.

FIG. 2 is a frontal view of the bandage depicted in FIG. 1 further having formed thereon opposed strips of adhesive layers and an absorbent mid-section for contacting with a wound.

FIG. 3 is a rear-view of the shrink-wrap adhesive bandage depicted in FIG. 2.

FIG. 4a is a perspective view of the leg of an individual depicting a shrink-wrap bandage of the present invention placed upon the knee thereof, the shrink-wrap bandage assuming a first expanded configuration.

FIG. 4b is a perspective view of the leg and bandage of FIG. 4a with a heat source being applied thereto, the latter causing the shrink-wrap bandage to assume a second, operative shrunken configuration.

FIG. 4c depicts the leg and bandage of FIGS. 4a and 4b, wherein the bandage has assumed a most-shrunken configuration.

FIG. 5a is a perspective view of a finger depicting a shrink-wrap bandage of the present invention positioned to be wrapped thereabout.

FIG. 5b is a perspective view of the finger and bandage of FIG. 5a with a heat-source being applied thereto, the latter causing the shrink-wrap bandage to assume a second, operative shrunken configuration.

FIG. 5c depicts the finger and bandage of FIGS. 5a and 5b wherein the bandage has assumed an operative shrunken configuration.

### DETAILED DESCRIPTION OF THE INVENTION

The detailed description set forth below is intended primarily as a description of the presently preferred embodiment(s) of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

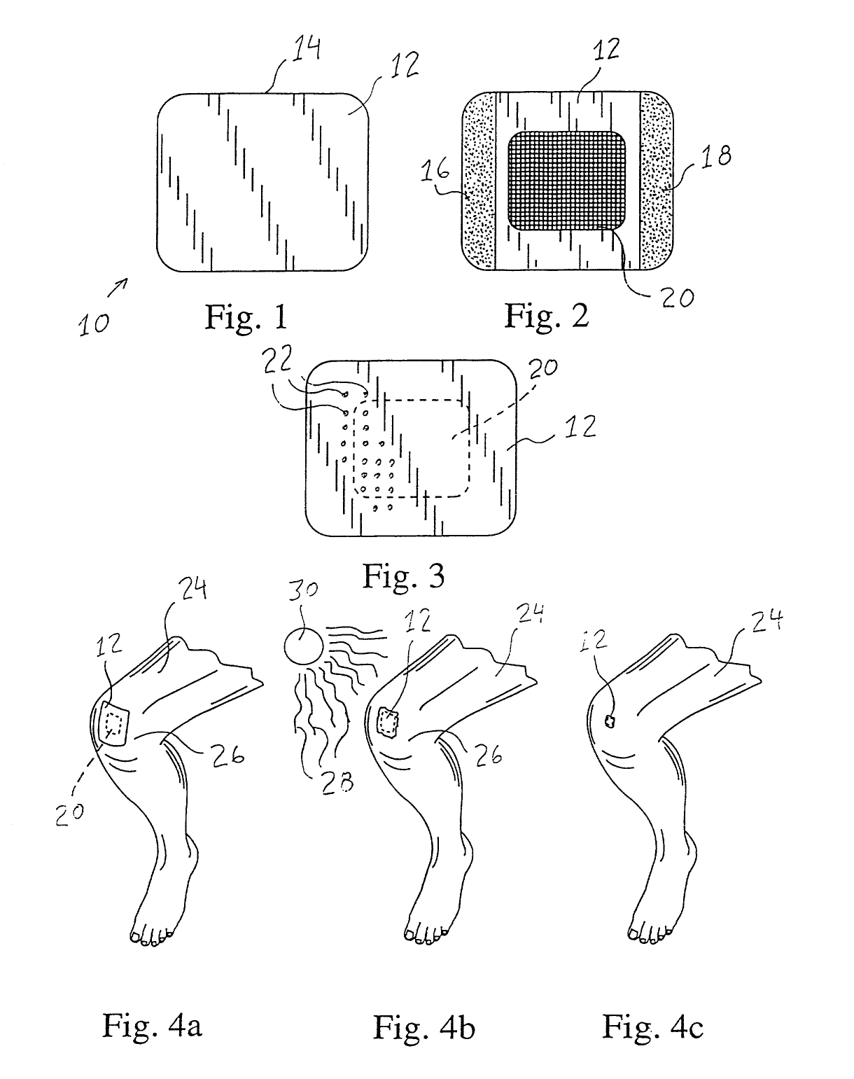

Referring now to the figures, and initially to FIG. 1, there is illustrated a shrink-wrap bandage 10 constructed in accordance with a preferred embodiment of the present invention. As illustrated, such bandage 10 comprises a sheet strip 12 of thermally-transitional material, namely, shrink film that may be formed and/or cut to have a desired peripheral edge 14. In this regard, it should be recognized at the outset that the bandage 10 of the present invention may be designed to take any of a variety of shapes and configurations as may be desired for applications upon various parts of the human body. Accordingly, the shape of the bandage 10 of the present invention should not be limited to the generally square or rectangular shape as depicted, but instead includes other substantially two-dimensional shapes such as circles, triangles, or other polygons, and also includes three-dimensional shapes such as a substantially cone-, thimble-, cylinder-, or sphere-shaped, or other irregular shape designed to accommodate a particular body part or area. In each case, the three-dimensionally shaped bandage preferably has an opening for receipt of the target area such as a finger or toe.

The sheet 12 of shrink film preferably comprises either polyvinyl chloride (PVC) or polyolifin shrink film which, as is well-known to those skilled in the art, are well-known and used widely for a variety of product packaging applications. Preferably, sheet 12 will be formed from a film of at least 50 gauge or greater, with 75 gauge shrink film being preferred. It should be understood, however, that shrink films having a lesser gauge may also be possibly utilized in the practice of the present invention.

In an alternative embodiment, the sheet 12 may be coated or laminated on one or both respective sides thereof with a layer of plastic or some other polymer film to insulate such sheet 12. In this regard, to the extent it is undesirable or unwarranted to allow the shrink film to come directly into contact with the user's skin, as discussed more fully below, such laminate or coating may serve as a barrier between the skin and shrink film. Such film or coating may take any of a variety of materials well-known to those skilled in the art, such as polyethylene, polypropylene, and the like.

Such shrink film sheet 12 is operative to undergo a shrinking size transition when subjected to temperatures of approximately 115 degrees Fahrenheit, although temperatures of 120 degrees Fahrenheit or higher may also be employed. As is well-known in the art, shrink films, such as PVC and polyolifin shrink films, can be manufactured to precise percentages of shrinking relative their expanded configuration when subjected to specified temperatures. For purposes of practicing the present invention, it is contemplated that any suitable shrink film should be formulated to undergo approximately 8-20% shrinkage when subjected to temperatures of approximately 115 degrees or higher for approximately two seconds or longer. In a more preferred embodiment, such shrink film should undergo approximately 12-14% shrinkage when subjected to approximately 115 degrees for a two to three second time frame.

Among such materials suited for application of the present invention include 500-degree shrink-wrap, which is commercially available and well-known in the art, that is formulated to shrink approximately 35% at 500 degrees Fahrenheit. Such material is available from a wide variety of sources, including Reynolds Metals Company of Richmond, Va., and distributed by AJM, Incorporated of San Leandro, Calif.

The bandage of the present invention may be manufactured of material having a specific transition temperature or temperature range, responsive to application of heat for a specific amount of exposure time or range of time, depending on a particular application. A single bandage may even comprise multiple materials, or a gradation variation of the same material, in each case with varying transition temperature and/or exposure time characteristics. For example, a thimble-shaped bandage for use on a digit may have a top portion having a higher transition temperature and/or exposure time requirement than the bottom open portion, thus facilitating the bottom open portion adhering sooner and more tightly to the base of a digit than the closed top portion adhering to the tip of the finger. This arrangement may be desirable to seal the fingertip, while at the same time avoid applying pressure to the fingertip.

Thus, a bandage may comprise shrink film comprises a first portion operatively transitional between the first expanded configuration and the second shrunken configuration in response to application of the transition source thereto for a first duration, and a second portion operatively transitional between the first expanded configuration and the second shrunken configuration in response to application of the transition source thereto for a second duration, wherein the first duration is different than the second duration.

Another example is that while a portion of the bandage 10 may transition from a first state to a second state in response to heat, a separate portion of the bandage 10 may not have a thermal transition property at all. The separate portion thus being a non-transitional portion.

As discussed more fully below, an important aspect of the bandages 10 of the present invention is the ability of the bandage 10 or portion thereof to undergo transition from a first expanded state to a second shrunken state in response to an appropriate transition source, such as a heat source of at least approximately 115 degrees Fahrenheit or temperature range, insofar as such temperature or range is substantially above normal human body temperatures, and above temperatures typically encountered in shipping and the like, and thus will prevent the same from prematurely or inadvertently transitioning. In this regard, in light of the fact that the shrinkage process is typically irreversible, it should be recognized that such transition temperature be engineered to remain at a sufficiently high level, relative to ordinary environmental temperatures, but at the same time not too high so as to cause damage to the skin and surrounding tissues during application, as discussed more fully herein.

Referring now to FIG. 2, there are shown additional optional elements to facilitate the functionality of the bandage 10 of the present invention. Upon the sheet 12 may be adhesive, such as in layers 16 and 18 as depicted. Such adhesives may take any of a variety of those known in the art. While the use of adhesive is considered optional in the practice of the present invention, such layers of adhesive 16, 18 may nonetheless be provided to further ensure secure attachment of the bandage 10 of the present invention about the wound site. To the extent such adhesive layers are utilized, however, it should be recognized that typically the same need only be applied in minimal amounts relative to conventional adhesive bandages, which consequently results in a bandage that is substantially easier and less costly to produce.

Such bandage 10 may further include one or more layers of gauze or absorbent material, such as 20, which are designed to absorb fluids from the wound and/or provide a cushioning, protective covering directly over the open wound. Such gauze or padding 20 may take any of a variety known in the art, including those typically employed with conventional adhesive bandages.

Referring now to FIG. 3, there is shown the backside view of the bandage depicted in FIG. 2. As per conventional adhesive bandages, the gauze or absorbent material 20 will preferably be centrally disposed upon the sheet or backing 12. Such sheet 12 may further preferably include perforations or apertures 22, as per conventional bandages, to enable air to access the wound, as is desired to promote the healing process.

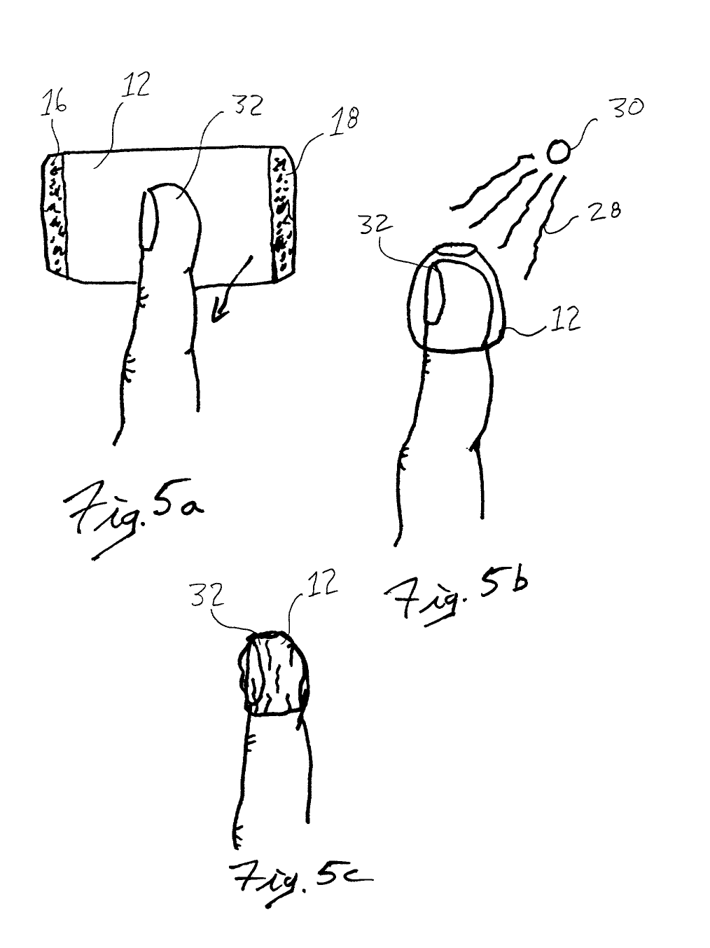

Referring now to FIGS. 4a-4c, and initially to 4a, there is shown a method by which a bandage of the present invention is deployed. As an example of how such bandage may be deployed, there is depicted a leg 24 showing the bandage placed upon the knee 26 thereof. As depicted, the sheet 12 is applied over the target area or wound (not shown) such that the gauze 20 is compressed thereagainst as per conventional bandages. As will be appreciated, the bandage is applied such that sheet 12 exists in its first expanded configuration.

Thereafter, as shown in FIG. 4b, heat 28, emanating from a heat source 30, is applied to the bandage such that the sheet 12 thereof transitions from its expanded configuration to its shrunken, operative configuration. As discussed herein, the specific amount, location, and duration of such heat 28 required to cause the sheet 12 of the bandage to transition from its first state to its second shrunken state, may vary depending on the material characteristics of the bandage 10. The heat 28 will preferably attain temperatures of at least approximately 115 degrees Fahrenheit. The source 30 from which such heat emanates may take any of a variety of conventional devices, including conventional hand-held hair dryers, heat lamps, and the like.

It is further contemplated that any source 30 sufficient to impart the necessary energy to the sheet 12 or portions thereof to enable the same to transition from its expanded configuration to its shrunken configuration will be well-suited for the practice of the present invention. Along these lines, it is contemplated that other energy sources, such as UV or infrared radiation, special lighting, or any of a variety of techniques known in the art may be deployed that are operative to cause shrink film sheet 12 to transition in the desirable manner.

Advantageously, as such sheet 12 transitions from its expanded configuration to its shrunken configuration, the same will substantially adhere to the skin or other target area about the wound sought to be protected. In fact, such shrinkage ultimately forms a custom fit about the wound that causes the bandage to stay thereon more securely than prior art adhesive bandages. Indeed, even to the extent minimal amounts of adhesive are utilized, as discussed above with respect to FIG. 2, such lesser amounts of adhesive substantially avoid the problems of prior art adhesive bandages where excessive amounts of adhesive are known to attract dirt and bacteria, as well as cause substantial pain when removed from the wound area. Of further advantage for certain embodiments is that the shrink film, and more particularly the backing sheet 12, is optionally water-resistant, and more durable and resistant to inadvertent removal when contacted with water or moisture.

In a more exaggerated depiction illustrated in FIG. 4c, the sheet 12 of the present invention is shown having undergone extreme shrinkage such that the same undergoes a substantial reduction in size from its first expanded configuration. Although typically not desired, such reduction in size is provided to illustrate the manner by which the bandage of the present invention transitions from its expanded configuration to shrunken configuration, the process by which advantageously causes the bandage of the present invention to adhere to the surface of the skin. In the illustrated example, it is contemplated that such extreme shrinkage can possibly maximize skin adhesion.

Referring now to FIGS. 5a-5c, initially to FIG. 5a, there is shown a finger 32 positioned to receive sheet 12 thereabout, the latter having two layers of adhesive 16, 18, as discussed herein. In this example, such sheet 12 may be radially wrapped about the finger 32 while in its expanded configuration as depicted by the figures. Alternatively, the bandage may initially be provided in a wrapped format with a substantially closed top and an open bottom. Recall such configurations have been described herein as cone-shaped or cylinder-shaped, for example. Such configurations may be individually packaged conveniently similar to flat bandages as is known in the art, wherein they pop up to their desired three-dimensional format upon removal from the packaging. Or they may be packaged in attractive three-dimensional wrapping for marketing or other efficiency considerations.

Upon application to the wound or target area, the sheet 12 is then in condition for subsequent shrinkage by application of heat as described herein. Again, although not required, adhesive 16, 18 may be provided to facilitate placement of the sheet 12 in the proper position and orientation relative to an anatomic structure, as seen in FIGS. 5a-5c in which the sheet 12 is placed about a finger 32.

Referring now to FIG. 5b, the sheet 12 positioned about finger 32 is subjected to heat 28 emanating from heat source 30. As discussed herein, heat source 30 may take any of a variety of conventional forms, as well as potential energy sources necessary to effectuate the transition of the sheet 12 from its first expanded configurations to its second operative configuration, and such quantity, duration, and location may vary depending on material characteristics of the sheet 12 or portions thereof.

After sufficient exposure to the heat 28 from heat source 30, as seen in FIG. 5c the resultant sheet 12 is shrunken about finger 32. In a typical embodiment of the present invention, the material characteristics of sheet 12 are preferably substantially uniform such that the transition temperature required to effect transition from a first state to a second shrunken state is at least approximately 115° Fahrenheit, and the duration of application of such heat is approximately two to three seconds, or even only approximately one second, or even any amount of time less than approximately 5 seconds. While in such configuration, the sheet 12 will tightly adhere radially about the finger 32 for as long as is practical.

As to the removal of the bandage of the present invention, the same may simply be peeled away from the wound site once sufficient duration has passed and/or the bandage needs to be changed. In this respect, such bandage may be easily peeled away with the fingers or tweezers. Advantageously, the bandage does not present any appreciable pain or discomfort when removed therefrom, especially as compared to removal of conventional bandages which typically use a relatively much greater amount of adhesive. As is known, prior art adhesive bandages and the like are known to cause substantial pain during their removal.

While certain embodiments are illustrated in the drawings and are described herein, it will be apparent to those skilled in the art that many modifications can be made to the embodiments without departing from the inventive concepts.

For example, although the examples described herein refer primarily to PVC or polyolifin as a preferred material for the shrink film, it is contemplated that any material or combination of materials may be used for the shrink film, which material has properties receptive to undergoing the desired transition when subjected to suitable levels of heat for suitable durations of time. Such materials may be known in the art as having such desired properties, or such materials or properties thereof may be later discovered.

As another example, the source of transition has been described herein primarily as heat or equivalent. Nonetheless, it is contemplated that other sources may effect the desired transition. For example, non-thermal elastomers (including siliconbased elastomers) or other materials known in the art may have material properties which transition upon exposure to environmental conditions substantially equivalent to room temperature and pressure, and/or exposure to elements in the air at quantities proportional to the naturally-existing quantities, such that upon removal of the bandage from a sealed package or the like, the bandage naturally transitions to its shrunken state within a tolerable time period. Such time period may be at least 5 seconds, or more preferably at least 10 seconds, or within a range of 5-10 seconds or 10-15 seconds. In this regard, the bandage may be unwrapped or unsealed, then applied at or about the target area, and will then transition in response to exposure to the air, thus forming the desired fit to the target area. Specific material properties may vary to accommodate temperature and pressure conditions native to particular regions, such as higher elevations and the like.

Accordingly, the particular combination of parts and/or steps described and illustrated herein is intended to represent only certain embodiments of the present invention, and is not intended to serve as limitations of alternative devices and methods which are within the spirit and scope of the inventive concepts described and claimed herein.

What is claimed is:

1. A shrink-wrap bandage for forming a covering about a wound comprising a sheet of shrink film operatively transitional between a first, expanded configuration and a second, shrunken configuration, said bandage being operative to substantially adhere to a target area about said wound as a result of said film transitioning from said expanded configuration to said shrunken configuration.

2. The bandage of claim 1 wherein said shrink film is selected from the group consisting of polyvinyl chloride and polyolifin.

3. The bandage of claim 1 wherein said shrink film is caused to transition from said first, expanded configuration to said second, shrunken configuration when subjected to a temperature of at least approximately 115 degrees Fahrenheit.

4. The bandage of claim 3 wherein said bandage transitions from said first, expanded configuration to said second, shrunken configuration in response to at least two seconds of exposure to said temperature.

5. The bandage of claim 3 further comprising adhesive formed thereon.

6. The bandage of claim 3 further comprising an absorbent pad thereon.

7. The bandage of claim 3 further comprising a plurality of ventilation apertures.

8. The bandage of claim 3, wherein said shrink film is selected from the group consisting of polyvinyl chloride and polyolifin, and wherein said shrink film has a gauge of at least 50.

9. The bandage of claim 8 wherein said shrink film has a gauge of at least 75.

10. A method for forming a covering of a target area such as a wound comprising the steps:
a) providing a shrink-wrap bandage, said shrink-wrap bandage comprising a shrink film operative to transition from a first, expanded configuration to a second, shrunken configuration in response to a transition source, said bandage being provided in said first, expanded configuration;
b) placing said bandage about said target area; and
c) applying said transition source to said bandage, causing said bandage to transition from said first expanded configuration to said second shrunken configuration.

11. The method of claim 10 wherein the transition source is a heat source, and said bandage operatively transitions from said first expanded configuration to said second shrunken configuration in response to a temperature of at least approximately 105 degrees Fahrenheit, said method further comprising the step of applying said heat source to said bandage at a temperature of at least approximately 105 degrees Fahrenheit.

12. The method of claim 11 wherein said bandage is operative to transition from said first expanded configuration to said second shrunken configuration in response to said heat source being applied thereto for a duration of at least two seconds, said method further comprising the step of applying said heat source to said bandage for at least two seconds.

13. The method of claim 11 wherein said bandage operatively transitions from said first expanded configuration to said second shrunken configuration in response to a temperature of at least approximately 115 degrees Fahrenheit, said method further comprising the step of applying said heat source to said bandage at a temperature of at least approximately 115 degrees Fahrenheit.

14. A shrink-wrap bandage for forming a covering about a wound comprising shrink film operatively transitional between a first expanded configuration and a second shrunken configuration in response to application of a transition source thereto, said bandage being operative to substantially adhere to a target area about said wound in response to said film transitioning from said expanded configuration to said shrunken configuration in response to said transition source.

15. The bandage of claim 14 wherein said shrink film comprises a first portion operatively transitional between the first expanded configuration and the second shrunken configuration in response to application of the transition source thereto for a first duration, and a second portion operatively transitional between the first expanded configuration and the second shrunken configuration in response to application of the transition source thereto for a second duration, wherein the first duration is different than the second duration.

16. The bandage of claim 15 further comprising a non-transitional portion.

17. The bandage of claim 15, wherein the shrink film is configured for application in a three-dimensional shape.

18. The bandage of claim 14 further comprising a non-transitional portion.

19. The bandage of claim 18 wherein the transition source is a heat source.

20. The bandage of claim 18, wherein the shrink film is configured for application in a three-dimensional shape.

21. The bandage of claim 14 wherein the transition source is exposure to substantially room temperature and pressure.

22. The bandage of claim 21, wherein the shrink film is operative to transition in response to exposure to substantially room temperature and pressure for a duration of at least ten seconds.
